(19) United States
(12) Patent Application Publication
(10) Pub. No.: US 2012/0172883 A1
(43) Pub. Date: Jul. 5, 2012

(54) REMOTE-CONTROLLED INTERNAL HYDRAULIC OSSEOUS DISTRACTOR

(76) Inventor: Ruben Fernando SAYAGO, Navojoa (MX)

(21) Appl. No.: 13/422,412

(22) Filed: Mar. 16, 2012

Related U.S. Application Data
(63) Continuation-in-part of application No. PCT/MX2010/000093, filed on Sep. 23, 2010.

(30) Foreign Application Priority Data
Oct. 5, 2009 (MX) MX/A/2009/010782

(51) Int. Cl. A61B 17/56

(52) U.S. Cl. 606/90

(57) ABSTRACT

The present invention relates to a hydraulic, electric, remotely controlled device, which will act as internal distractor to correct spinal deformitites, and/or to lengthen long bones in humans. The object of the present invention is to provide an internal distractor that is totally different from those currently in existence on the market and one which successfully solves the problems and improves the results achieved with distracters and devices currently used for this purpose, but which have limited results, and many problems inherent in current devices. This object will be achieved by virtue of the novel design thereof and the way in which the distractor operates, which is likewise novel, and because the distractor has features that combine the advantages of various devices and techniques in existence on the market for the same purposes.

### RELATED APPLICATION INFORMATION

This application is a continuation-in-part of International Application PCT/MX2010/000093 filed 23 Sep. 2010 and entitled “REMOTE-CONTROLLED INTERNAL HYDRAULIC OSSEOUS DISTRACTOR”, which was published on 14 Apr. 2011, with International Publication Number WO 2011/043638 A2, and which claims priority from Mexican Patent Application MX/a/2009/010782, filed 05 Oct. 2009.

### BACKGROUND OF THE INVENTION

Currently, deformities correction of spinals such as scoliosis or kiphosis is made by various techniques either chirurgical or non-chirurgical, or a combination of both. The use of corset is comprised in non-chirurgical techniques having a limited correcting effect, and its greatest efficiency is stopping or preventing the development of an existing curve. Chirurgical techniques comprise locating bars of different thickness (¼, 3/16 inches) and different material such as steel, titanium or the combination of other alloys whenever they are biocompatible, plates are also used. Bars are fixed to the spinal through transpedicular screws, hooks or wires, and plates are generally fixed to the spinal through transpedicular screws. This technique has limitations such as only distracting vascular and neurological elements from the spinal. Therefore, if the deformity is minor, the correction is accomplished at 100%, but if the deformity is moderate or severe, very rigid or structured, a partial correction is accomplished thereby causing several reoperations. There are further external distractors consisting of one or two threaded bars of different length (according to some necessities) and different thickness (¼, or ½ inches), to which two cubes of the same material (2 minimum) are attached. These cubes have a central hole with a larger diameter than the bars', where cubes enter and are movable throughout the bars via two nuts fixed to each edge of the cube, and being of the same measurement of the bar's rope. The cubes have also the same two-smallest holes (4 to 6 mm of diameter) where nails or screws enter and are fixed from the outside of the body onto the pedicles of the spinal bodies (spinal bodies of the deformity edges to be corrected). For example, a scoliosis deformity comprises from T8 vertebral body to L4, therefore, transpedicular screws or nails will be located only on those vertebral bodies (two nails or screws per each vertebral body as vertebral bodies just have two pedicles). By means of the nuts moving the cubes, a separation of both cubes is made achieving distraction and correction of the deformity. Screws or nails are fixed to the cubes via oppressors in the cubes, the correction is achieved in the way abovementioned, but the drawback is the external distractor. That is to say, the patient carries it outside his/her body, and it is fixed to the spinal by means of the nails and screws. And as the velocity of the correction should be slow, at 1 mm per day, for preventing neurological damage to the patient, and granting a suitable blood supply of the spinal in the zone being distracted, this implies carrying on this distractors for long periods (months) with all the inherent problems such as uncomfortableness, pain, infection, due to the sharp-pain going through the skin up to the spinal.

As per lengthening long bones, the technique used is made with external distractors, working in the same way as the distractors used for the spinal. Such distractors may have different forms or may be of different models (mono-polar, bi-polar, with semi-circular or circular rings, etc.) but are variants of the same model. The drawbacks of such, apart from being inside the spinal, are disuse osteoporosis, articulation contracture adjacent to the bone which is lengthened, as well as annulment on the place of lengthening.

With the purpose of improving the technique and the results either in the correction of the spinal deformities or lengthening long bones, the invention of a device working as internal distractor and intended to be protected with the application herein was thought, as it is a an internal apparatus. That is to say, the device is to be mounted and located inside the patient thereby achieving a distraction via an hydraulic system, and handled via remote control, while the patient lives his/her normal life, respecting the velocity of distraction of 1 mm per day, but, as it is internal, it will not cause the drawback of the current methods.

### DESCRIPTION OF THE INVENTION

The characteristic details of this novel device are clearly presented in the following description, and in the figures enclosed herein, as well as an illustration of the drawings having the same reference signs to indicate the parts and the figures presented.

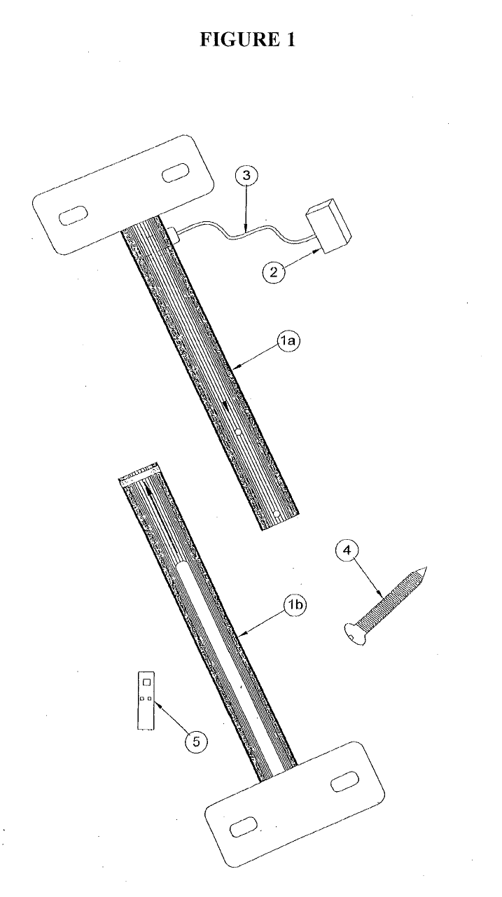

FIG. 1 is a conventional perspective of the T-form device.

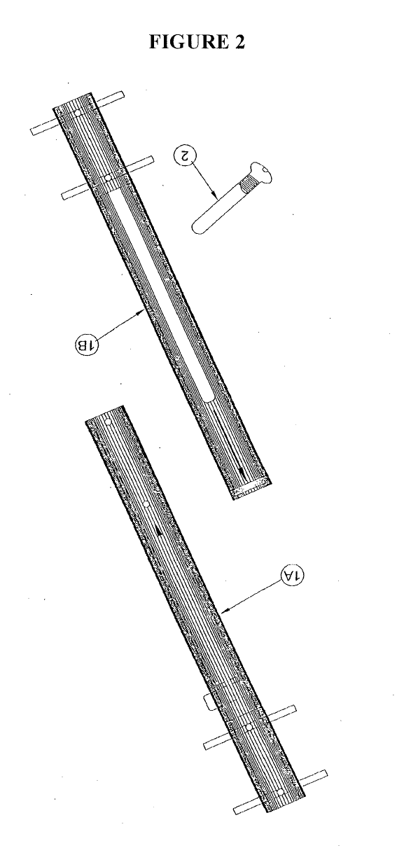

FIG. 2 is a conventional perspective of the I-form device.

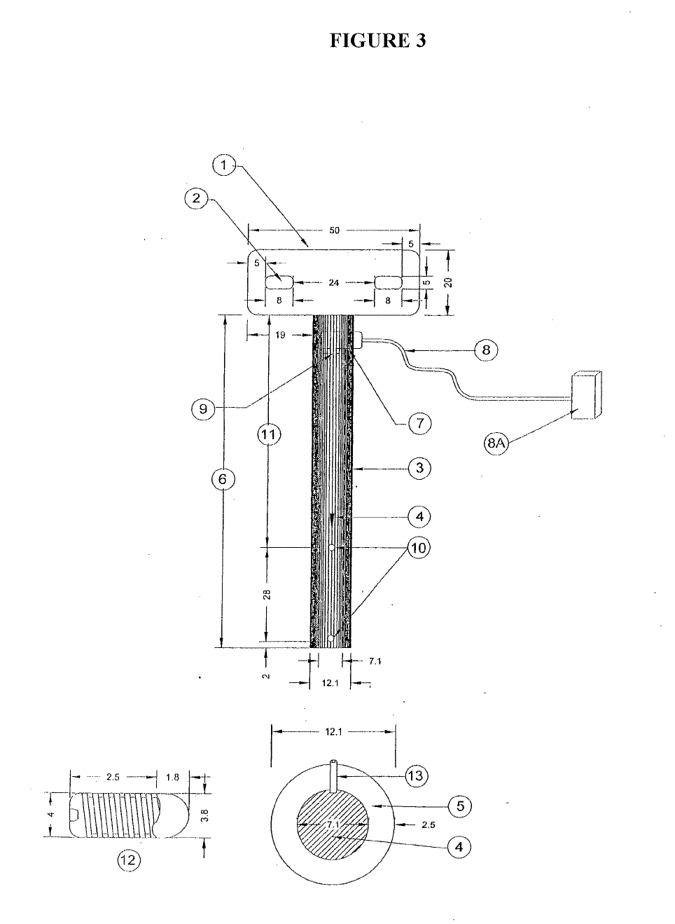

FIG. 3 is a perspective of part A of the T-form device.

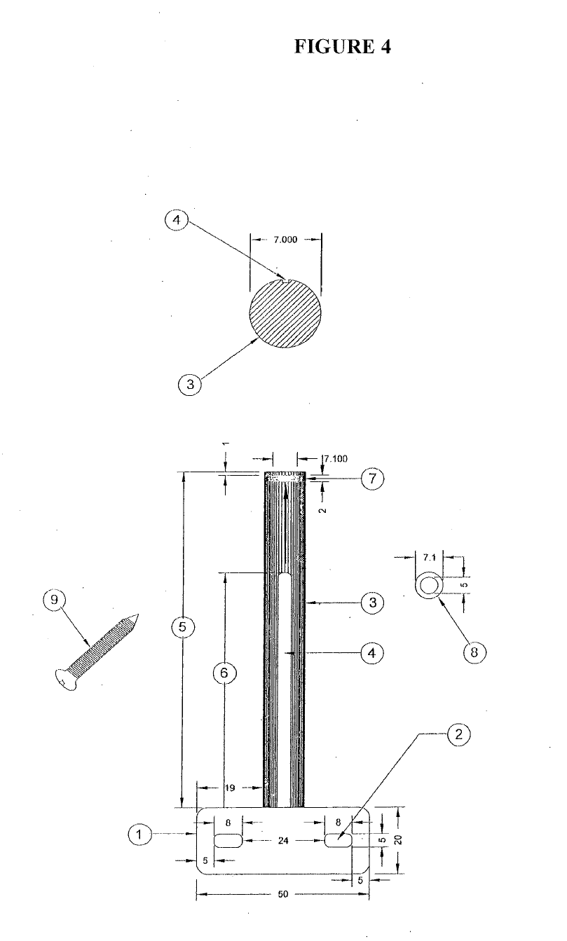

FIG. 4 is a perspective of part B of the T-form device.

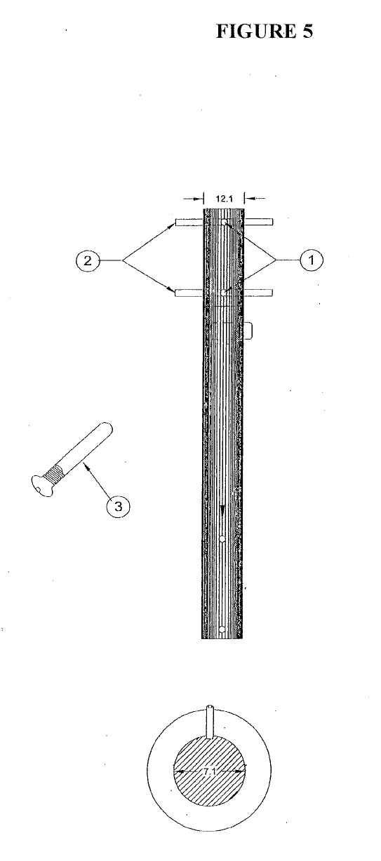

FIG. 5 is a perspective of part A of the I-form device.

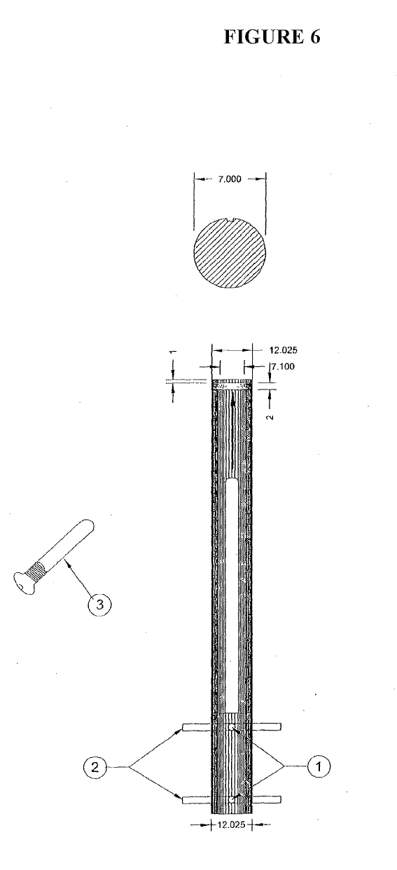

FIG. 6 is a perspective of part B of the I-form device.

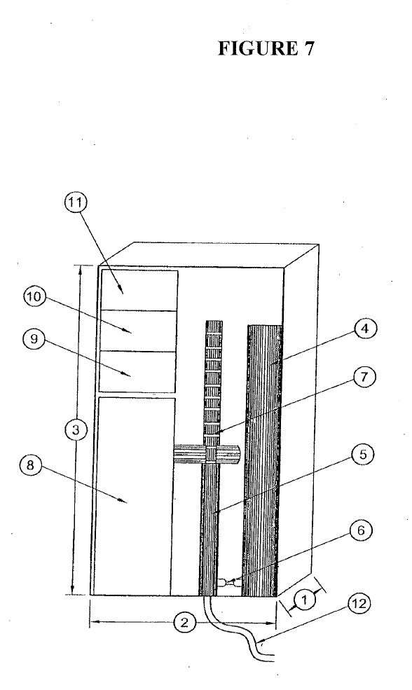

FIG. 7 is a perspective of the injector apparatus.

With reference to the figures: the device includes two parts FIG. 1 (1a, 1b), FIG. 2 (1A, 1B) (a part A and a part B), an injector apparatus (2) FIG. 1, 4 bolts or 4 screws whatever the case is, FIG. 1 (4) FIG. 2 (2) and radiofrequency remote control (5) FIG. 1, the material should be preferably made of titanium 6, aluminum 4, vanadium, or biocompatible steel, which are assembled and sliced one another, part A will be connected through a hose with the injector apparatus (3) FIG. 1, the hose may have a variable diameter as well as its length but preferably of 4 mm. Each part of the device may be each part in T-form (A and B), FIG. 1 (1a, 1b) which is to be used to correct the spinal vertebral deformities, or in I-form FIG. 2 (1A, 1B), which is to be used for lengthening long bones as FIGS. 1, 2, 3, 4 and 5 show.

Part A (T-form) will be a T and which size of horizontal axe (1) of the T FIG. 3 will be of 50 mm×12.1 mm×20 mm. This axe will have 2 oval holes for anchorage to the spinal of 5 mm×8 mm on each edge of the horizontal axe, as shown in FIG. 3 (2), the vertical axe (3) will be a hollow cylinder (4) of 12.1 mm of external diameter, and an internal diameter of 7.1 mm, living an empty wall of the cylinder of 2.5 mm (5), the length of this cylinder will be variable (6), according to what it is necessary 18 cm, 20 cm . . . 35 cm (but of the same length as part B), at 2 mm from its base or union to the horizontal axe. There will be a threaded hole of 4 mm (7) which will connect the hollow cylinder, and will serve as connection to the hose of the injector apparatus (8, 8a), and where the liquid will enter and make function the device. Immediately over the connecting hole, and inside the hollow cylinder will be a one-way valve (9) that, as its name defines it, will only allow the liquid entrance to the hollow cylinder, but not the exit of such. At 2 mm from the vertical axe top will be a hole of 4 mm of threaded diameter, aligned with this, there will be another hole of 4 mm of diameter (10) which will be located at 70% of length from the vertical axe of the T (11). This measure will be taken from the vertical axe union with the horizontal axe of the T (11), via these holes, a pair of screws will enter (12) (one on each hole) of 4 mm of diameter, and 4.3 mm of length with hexagonal head of 3.5 mm and with a rope of 2.5 mm which will correspond to the cylinder wall (13). The top of this screw (12) will be of 3.8 mm of width×1.8 mm of length. The two screws (12) located in the holes (10) of the cylinder, will be assembled to the slot in part B (4), FIG. 4, and will have 2 objectives, 1) preventing the rotation between both parts (part A and part B), and 2) the sliding stop of the part B distraction over part A, as slot of part B will end at a point of 70% of length of the vertical axe of the T as shown in FIG. 4 (4, 6).

Part B (T-form) will be a T-form, FIG. 4, and the horizontal axe of such (1, 2) will be identical to part A horizontal axe, the vertical axe of the T will be a solid cylinder of 7 mm of diameter (3), and which length (5) will be variable according to some necessities (but of the same length as part A). As abovementioned, there will be a slot in the vertical axe of the T (4) of 4 mm of width and 2 mm of depth, and will start at the vertical axe union with the horizontal axe, and will end at a point that will correspond to 70% of the vertical axe length of the T (6). At 2 mm of the top of the vertical axe there will be a groove (7) throughout the circumference of the cylinder of 1 mm of width and 2 mm of depth, on which an o-ring will be located (8), which will serve as sealing ring which will prevent the spillage, or exit of the hollow cylinder liquid. The anchorage of the spinal will be made via 4 screws, FIG. 1 (4) and FIG. 4 (9), two at each edge of the assembled device, with a variable diameter and length according to what is necessary, with an hexagonal head of 4.5 mm, that will pass through the holes of the horizontal axe of each part (A and B), FIG. 3 (2), FIG. 4 (2), and will be inserted in the pedicles of the vertebral bodies chose to achieve the deformity correction.

The device's part A in form of I will only differ from T-form part A described in the form FIG. 5 and in the holes being of 4 mm of diameter, circular, and aligned longitudinally at the base as shown in FIG. 5 (1), I-form part B will only differ from part B in the form and the holes will be longitudinally aligned at the base and will be circular with a diameter of 4 mm as shown in FIG. 6 (1). The distractor will be intramedullary located in the long bone to be lengthened, the anchorage in the long bone will have 4 bolts, 2 on each edge of the assembled device, FIGS. 5 and 6 (2) of 4 mm of diameter, variable length according to what is needed, and hexagonal head of 4.5, and rope of 3 mm of length followed by the hexagonal head, FIGS. 5 and 6 (3), this bolt will cross through the other bone cortical.

The injector apparatus will be a metallic box, FIG. 1 (2), that will have the following dimensions 2 cm×7 cm×9 cm, as shown in FIGS. 7 (1, 2 and 3). The injector apparatus will contain 2 cylinders, FIGS. 7 (4 and 5) united one another through a tube of 2 mm of diameter and a one-way valve, FIG. 7 (6), one of the two cylinders will be the container of the liquid to be injected and will have the following dimensions 1.8 cm of diameter per 7 cm of length, FIG. 7 (4), the other will be the injector cylinder and will have the following dimensions 5 cm of diameter per 2 cm of length, FIG. 7 (5), which in syringe form will have a piston, and a bar attached to this piston will be indented with a thickness of 6 mm and 3 cm of length, FIG. 7 (7), and will be assembled to an electric servomotor of 7.7 kg-cm of torque FIG. 7 (8). It will also comprise 4 batteries to operate the electric motor, FIG. 7 (9). The electric circuits, FIG. 7 (10), and a radiofrequency sensor, FIG. 7 (11), which will receive the signal from a radiofrequency remote control, FIG. 1 (5), to operate the servomotor, from this box a hose of 4 mm will exit which will be attached to the injector cylinder, FIG. 7 (12), and will be connected to part A of the device, in such a way that the injected liquid will pass from the container cylinder to the injector cylinder, from this one to the hose, and from this one to the hollow cylinder through the one-way valve, from part A of the device.

The device will operate as follows: part A and B assembled, as well as part A connected to the hose of the injector apparatus. Such apparatus will receive a signal from the remote control which will make function the electric motor of the injector apparatus, which connected to the bar holding the injector cylinder piston will make 2 movements. The first one will be picking up the container cylinder liquid, and thus filling the injector cylinder, this liquid will pass the container cylinder to the injector cylinder through the tube uniting them, and the one-way valve will only allow the passage of the container cylinder liquid. The second movement will be injecting the injector cylinder content, this liquid will close per pressure gradient the one-way valve that unites both cylinders, thus making the liquid pass through the hose uniting the injector apparatus to part A of the device, and will enter the cylinder through the one-way valve, the liquid will move or slide part B over part A, achieving distraction. The amount of liquid required for distracting or enlarging the device of 1 mm is 0.039 ml.

The design and form of this distractor constitutes the novelty of this device. The design allows, because of its dimensions, to be located in an internal way inside the patient. The anchorage inside the body, either in the spinal to correct it, or in the long bones to lengthen them, will be firm by its dimensions of the bolts or screws that will be employed for such effect. The location of the injector apparatus will also be inside the patient and it is possible because of its dimensions. The way in which the distractor functions is also a novelty, because it can be operated from the distance thanks to the use of remote control, allowing the patient to live a normal life while the device operates daily. It is important to mention that the remote control will be programmed to send just one signal within 24 hours, thereby granting that, when the patient may get desperate, he/she will not be able to make various shots causing damage to himself/herself.

Below it is shown the way the internal distractor is obtained and which has the following characteristics:

a) The device will be located inside the patient, while with existing methods, there exist limitations abovementioned such as limited correction of the deformity at the moment of the surgery, due to the potential danger of causing neurological or vascular damage to the patient, or the uncomfortableness of carrying the external distractor with the inherent problems, uncomfortableness to the patient, infections, etc.

b) As distraction is very slow, at 1 mm per day, it will not cause pain to the patient, otherwise the distraction will be stopped for some days, being later on restarted, having the sufficient time to achieve the deformity correction at 100%.

c) Another important aspect of this novel device is that the velocity of distraction will be regulated by programming the remote control for making it emit just one signal to the injector apparatus within 24 hours, as well as the amount of liquid to achieve the distraction of 1 mm per day that will be of 0.039 ml. Current models of external distraction allow the desperate patients to rotate the nuts beyond the limit, moving the cubes that at the same time make possible the distraction, and thereby causing several vascular or neurological lesions.

d) With reference to the bone lengthening, as the device will be intramedullary located, it will serve as strut to the long bone giving it strength and preventing it from the annulment of the lengthening place. Apart from the fact that the patient is able to live his/her life normally and is able to walk and use the bone being lengthened, there will not be any disuse osteoporosis, or contracture of the adjacent articulations of the bone being lengthened.

Accordingly to the abovementioned, it is certain that these internal distraction characteristics haven't been achieved by any other artifact or device, and they include the characteristics of an internal distraction. Other characteristic is that the patient may be able to live his/her life normally while the distraction is achieved correcting a spinal deformity, or lengthening long bones of his/her body.

1. An internal distractor system used to correct spinal deformities and/or lengthen long bones in humans, which will be surgically located inside the patient, such system comprises:
2 cylindrical parts, one hollow A, one solid B, that operate assembled and slide telescopely one another, such system being in T-form for correcting spinal deformities, or I-form for lengthening, long bones;
an injector apparatus containing enough liquid to achieve the complete distraction of the device and that will be attached to the hollow part A via a hose, which will be connected to a hole located at the base of part A, and
a radiofrequency wireless remote control;
both part A and B as well as the injector apparatus being adapted to be located inside the patient, and the radiofrequency remote control being adapted to will be located outside the patient;
the injector apparatus being a metallic box made of the same material as part A and B of the device, such injector apparatus containing 2 cylinders, a piston attached to an indented bar attached to an electric servomotor, electric power batteries, electric circuits, a radiofrequency sensor, and a hose attached to the injector cylinder by an edge, which outside the box is attached to part A of the device;
one cylinder of the injector apparatus being will be a container of the liquid to be injected, and the other one being the injector cylinder of the liquid, both cylinders united by a tube and a one-way valve, that will only allow the passage of the liquid from the container cylinder to the injector cylinder that by its dimensions will grant that the amount of injected liquid be specific to achieve the distraction of about 1 mm; in each syringe of such cylinder to the hollow part A of the distractor, the injector cylinder comprises a piston attached to an indented bar assembled to the servomotor cogs; the distractor system being operable when the radiofrequency sensor of the injector apparatus receives a radiofrequency signal of the remote control outside the patient, and this received signal makes the servomotor operate in two movements, the first movement of the injector cylinder piston aspirating liquid from the container cylinder, and once the injector cylinder is full of liquid, the second movement of the servomotor injecting such liquid, closing the one-way valve located in the tube uniting the two cylinders, thereby making the liquid flow to the hose connected to part A of the device, achieving the injection of the injector apparatus liquid to hollow part A sliding solid part B assembled to hollow part A because of the effect of the injected liquid,
whereby the liquid injected to hollow part A of the device will support the distraction achieved, thanks to its design and to the one-way valve comprised in hollow part A of the device, that will only allow the entrance of the liquid from injector apparatus to part A, the remote control being programmed to emit just one signal to the injector apparatus within 24 hours.

2. Internal distractor system of claim 1 adapted to be internally implanted to the patient by a surgical process, with the spinal instrumentation or centre-bone marrow nails techniques and be anchored either to the spinal via transpedicular screws, or to the long bone to be lengthen via bolts, and the injector apparatus being adapted to-be located in some place of the lumbar fascia, when correcting a spinal deformity, or when lengthening a long bone of the abdomen, and connected to the hose of the injector apparatus to part A of the device.

3. Internal distractor system of claim 1, in T-form which is adapted to correct spinal deformities by the anchorage thereto through the holes of the horizontal axis of the T which serve to locate transpedicular screws.

4. Internal distractor system of claim 1, wherein part A of the device contains a first hole in the vertical axe of the T located at 70% of the vertical axe length, taking this measurement from the union of the vertical axe with the horizontal axe of the T, and in I-form of 1 cm of distance from a connecting hole to the injector apparatus hose, through which-hole a screw may be inserted to couple to the anti-rotation slot of solid part B of the device, whereby it will serve to prevent rotation between part A and part B assembled and further, serve as maximum stop of the device distraction.

5. Internal distractor system of claim 4, wherein part A of the device will contain a second hole in the vertical axe of the T, that will cross the cylinder wall located at 2 mm of the cylinder top, and aligned to said first hole, through which a screw may be inserted to couple serve to prevent rotation between part A and part B assembled, further, it will serve as maximum stop of the device distraction.

6. Internal distractor system of claim 1, useful to correct spinal deformities involving different levels or spinal bodies, whereby once part A and part B are assembled, they will be from 18 cm, to 35 cm, thereby being adapted to be anchored to the normal spinal bodies located at the edges of the deformity, distracting deformed spinal bodies, that will be 70% of assembled device's length, and such distraction being achieved by the injected liquid to part A, and by the screw entering the hollow part A and further entering the anti-rotation slot of solid part B of the device achieving a great lengthening of the long bones.

7. Internal distractor system of claim 1, wherein solid part B contains in one of the edges two holes for locating screws, which serve to anchor the bone to be treated; as per T-form these screws will be located in the horizontal axe of the T, and as per 1-form, these screws will be longitudinally located in one of the edges, finally locating the bolts.

8. Internal distractor system of claim 8, wherein solid part B contains an anti-rotation slot in the vertical cylinder of 4 mm of width and 2 mm of depth, and commencing at the union of the vertical axis with the horizontal axis, ending at a point corresponding to 70% of the vertical axe of the T, through this slot two screws will enter coming from the holes of hollow part A, and will serve to prevent rotation between part A and part B assembled, and will also serve as maximum stop of the device distraction.

9. Internal distractor system of claim 8, wherein solid part B will be located at 2 mm of the vertical axis top, a groove throughout the circumference of the cylinder of 1 mm of width and 2 mm of depth, in which an o-ring may be located, which will serve as sealing ring preventing the spillage or exit of the hollow cylinder liquid.

10. Internal distractor system of claim 1, wherein said injector apparatus has dimensions of 2 cm×7 cm×9 cm.

11. Internal distractor system of claim 1, wherein the container cylinder dimensions are 1.8 cm of diameter×7 cm of length.

12. Internal distractor system of claim 1, wherein the injector cylinder dimensions are 0.5 cm of diameter,×2 cm of length.

13. Internal distractor system of claim 1, in I-form adapted to lengthen long bones, which by its form will be intramedullary located in the bone to be lengthened, and by longitudinally locating the bolts in the aligned holes, for such effect, the distractor being adapted to be fixed to the bone
